(19) United States
(12) Patent Application Publication
(10) Pub. No.: US 2026/0041580 A1
(43) Pub. Date: Feb. 12, 2026

(54) SLEEP TRAINING SYSTEM

(71) Applicant: Anton Shvydkyi, Boiarka (UA)

(72) Inventor: Anton Shvydkyi, Boiarka (UA)

(21) Appl. No.: 19/296,855

(22) Filed: Aug. 11, 2025

Related U.S. Application Data
(60) Provisional application No. 63/681,781, filed on Aug. 10, 2024.

(51) Int. Cl. A61F 5/56

(52) U.S. Cl. CPC A61F 5/56

(57) ABSTRACT

A sleep training device is configured to detachably attach to a garment to position the sleep training device on a back of a person during sleep to prevent the person from rolling onto their back during sleep. The sleep training device may include clips to attach to the garment. The sleep training device may include a first component that is configured on an inside surface of the garment and a second component configured on an outside surface of a garment and the two components may detachably attach across the garment to be retained to the garment. The exterior component may actuate to couple to the interior component. The interior and exterior components may comprise a magnetic component to magnetically couple the two components together.

### CROSS REFERENCE TO RELATED APPLICATIONS

This application claims the benefit of priority to U.S. provisional Ser. No. 63/681,781 , filed on Aug. 10, 2024; the entirety of which is hereby incorporated by reference herein.

### BACKGROUND OF THE INVENTION

### Field of the Invention

The invention relates to a sleep training system that employs a sleep training device that is detachably attachable to a garment to protrude from a back of a person while they sleep to prevent them from rolling onto their back.

### Background

Many people snore or snore louder when sleeping on their back. There exist devices to position a protrusion on a person's back to prevent them from rolling onto their back while sleeping. These devices typically have straps that extend around a person's torso and/or over their shoulders to secure the device in a proper position on their back. These straps can get twisted or pull and pinch a person as they sleep and are uncomfortable.

### SUMMARY OF THE INVENTION

A sleep training device is configured to detachably attach to a garment to position the sleep training device on a person's back during sleep to prevent the person from rolling onto their back during sleep. The sleep training device may include clips to attach to the garment. The sleep training device may include a first component that is configured on an inside surface of the garment and a second component configured on an outside surface of a garment and the two components may detachably attach across the garment to be retained to the garment. The exterior component may actuate to couple to the interior component. The interior and exterior components may comprise a magnetic component to magnetically couple the two components together.

An exemplary sleep training system employs a sleep training device configured for detachable attachment to a garment by clipping an exterior component such as an exterior clip component to an interior component. The interior component is configured inside the garment, and the exterior clip component is configured on an outside of said garment and is actuated to an open position and clipped onto the interior component. A person may actuate the exterior clip component to an open configuration by hand and retain the interior component inside of the garment. They may then attach the exterior clip component to the interior component to secure the sleep training device to the back portion of a garment. The sleep training device creates a protrusion from the person's back to prevent them from rolling onto their back. As a person starts to roll on their back, the sleep training device presses on the person's back to stop them from rolling onto their back.

The sleep training device may be spherical in shape or cylindrical in shape, wherein the pressure on the person's back is increased as they continue to attempt to roll onto their back. The body of the sleep training device may be made of a resilient material, such as an elastomeric material that can be compressed and deformed by a compressing force and then return to an original shape after removal of the compressing force. A resilient and elastomeric material may be foam, such as a polyurethane foam.

An exemplary sleep training system includes a sleep training device having an interior component with a flange and an exterior clip component configured to actuate to open a flange aperture on a flange interface surface to couple the exterior clip component to the flange of the interior component.

The interior component has a flange configured to receive flange receiver of the exterior clip component. A flange may extend from the interface surface of the interior component and may extend a flange extension distance. A flange may be configured a flange offset distance from the clip interface surface of the interior component. The flange may be configured on the extend end of the flange hub. The interior component extends to a distal surface opposite the interface surface and configured to contact a person's back when the sleep training device is detachably attached to a garment and said garment is donned for sleeping. The distal surface is a pressure surface or surface that applies pressure to the person's back while sleeping. The distal surface may extend along a radius of curvature, and the interior component may be spherical in shape along the distal surface. An interior component may have a sphere shape, such as a partial sphere or half sphere.

The exterior clip component may include a plurality of clip portions that are actuated to attach the exterior clip component to the interior component, such as two, three, four, five or more. The clip portions may be coupled together by clip springs and may have a clip handle to enable easy manipulation to move the exterior clip component from a closed configuration to an open configuration. The handles may be squeezed or actuated inward toward a centerline or center point of the exterior clip component to open a flange aperture for coupling to a flange on the interior component.

The exterior clip component has a flange receiver, that may be a recess or groove within the flange aperture. The clip portions may be coupled together by clip springs that extend between adjacent clip portions to force the clip portions and exterior clip component to a closed position, to retain the exterior clip component to the interior component.

The clip portions may have a hinge protrusion on a first side and a hinge receiver on a second side of the engagement surface of the clip component. The hinge protrusion of clip portion may be configured for coupling with the hinge receiver of an adjacent clip portion to form a hinge. Also, each clip portion may have a spring receiver, such as a recess, flange or aperture to retain the clip spring. The spring receiver of clip portion may be an aperture for insertion of a first extended end of the clip spring and spring receiver may be an aperture for receiving the second extended end of the clip spring.

The clip portions of the exterior clip component may be hingeably engaged by hinges and have clip springs coupling together the clip portions. The exterior clip component may be in a closed configuration when the springs are extended and configured in an open configuration when the springs are compressed or flexed to bring each of the engagement surfaces proximal to the extended end of the clip portions toward each other.

The exterior clip component may also have a spherical shape wherein each of the clip portions from a wedge shape of a half spherical shape. When the interior component and the exterior clip component are attached, the sleep training device may be a full spherical shape having a diameter of about 60 mm or more, about 70 mm or more, about 80 mm or more, about 90 mm or more, about 100 mm or more and any range between and including the diameters provided.

An exemplary sleep training system may include a sleep training device having an interior component with a flange and an exterior clip component with two clip portions that are configured to actuate to open a flange aperture on a flange interface surface to couple the exterior clip component to the flange of the interior component.

An exemplary sleep training system may include a sleep training device having an interior component with a flange and an exterior clip component with an actuator such as an actuator button for attaching and detaching the exterior clip component to the interior component. The actuator button may be coupled with latches that are actuated to an open position when the actuator button is pressed.

The actuator button moves an actuator interface that interfaces with the lathes to move from a closed to an open position. A return spring moves the actuator button back to a ready position. The actuator button moves into the exterior clip body when actuated to move the latch to an open position.

An exemplary sleep training system may include a magnetic sleep training device that includes a first component having a first magnetic component and a second component having a magnetic component configured to magnetically couple with the magnetic component of the first component. The first component body and the second component body may have a recess to receive the respective magnetic component. The magnets may be configured on the interface surface of the first and second components respectively. The distal surface of the first and second components may be rounded and each of the first and second components may have a spherical shape, a half of a sphere that forms a full sphere when attached by the magnets on the interface surfaces. A stabilizing component may be configured between the first magnetic component and the second magnetic component to stabilize the connection and prevent sliding and to also make it easier to decouple the first and second magnets from each other to remove the magnetic sleep training device from a garment. Also, the first component body and second component body may be made of a resilient material, such as a foam, such as a polyurethane foam and may be a high density polyurethane foam.

An exemplary magnetic sleep training device may include a first component having a magnetic component and a second component having a magnetic component configured to magnetically couple with the magnetic component of the first component. The first component may have an interface component and the second component may have an interface component configured to retain the respective first magnet and second magnet. The magnetic component may be retained in the first interface component by a fastener and the first interface component has a recess for receiving the magnetic component therein. The first component body may also have a recess for receiving the protrusion from the first interface component. The first interface component and second interface component form the respective interface surfaces of the first and second component and make it easier to detach the first component from the second component. The first and second interface components may form a decoupling gap that enables a person to insert their finger to pry apart the two components to detach the magnetic sleep training device from a garment. A magnetic component may be a magnet, such as a permanent magnet, or a material that is magnetically attracted to a magnet, such a steel or other metal that is attracted to a permanent magnet.

A sleep training device may be cylindrical in shape and have a length from a first end to a second end of about 50 mm or more about 75 mm or more, about 100 mm or more, about 150 mm or more, or even up to about 200 mm, and any range between and including the length values provided. The cylindrical sleep training device may have a diameter of about 50 mm or more, about 75 mm or more, about 100 mm or more, about 150 mm or more, about 200 mm or more and any range between and including the diameter values provided. Clips may be configured on either end of the sleep training device and may be configured on the extended ends of clip strap so that the strap is coupled to the sleep training device proximal to the center of the respective end. The sleep training device may be inflatable having an inflation port that fills a bladder. A cover may be configured around the bladder for comfort and may include a fabric, or a flocking material that coupled to a bladder.

The summary of the invention is provided as a general introduction to some of the embodiments of the invention and is not intended to be limiting. Additional example embodiments including variations and alternative configurations of the invention are provided herein.

### BRIEF DESCRIPTION OF SEVERAL VIEWS OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a person attaching a sleep training device onto a garment by clipping an exterior clip component to an interior component.

FIG. 2 shows the sleep training device attached to the back portion of a garment and is protruding from the person's back.

FIG. 3 shows a person sleeping on their side with the sleep training device of the sleep training system coupled to the back portion of a garment.

FIG. 4 shows the person in FIG. 3 rolling onto their back, wherein the sleep training device presses on the person's back to stop them from rolling onto their back.

FIG. 5 shows a perspective view exemplary sleep training system including a sleep training device having an interior component with a flange and an exterior clip component configured to actuate to open a flange aperture on a flange interface surface to couple the exterior clip component to the flange of the interior component.

FIG. 6 shows a side view of the sleep training device shown in FIG. 5 having a flange configured on an extended end of a flange hub and having a flange extension distance.

FIG. 7 shows a side view of the sleep training device shown in FIG. 6 with the exterior clip component opened about the hinge and configured over the interior component.

FIG. 8 shows a perspective view of an exemplary sleep training system including a sleep training device configured in an attached configuration with the exterior clip component detachably attached to the interior component.

FIG. 9 shows a perspective interface view the exterior clip component having a flange aperture and flange receiver.

FIG. 10 shows a top view of the exterior clip component of FIG. 8 having three clip portions coupled together by clip springs.

FIG. 11 shows a top view of the exterior clip component of FIG. 9 now in an open configuration with each of the three clip portions actuated to an open position to open the flange aperture.

FIG. 12 shows a top view of the three clip portions having hinge protrusions and hinge receivers for the hinge protrusions to form hinges for actuation of the exterior clip component from a closed configuration to an open configuration for attachment to the interior component.

FIG. 13 shows a perspective view of an exemplary clip spring having a hinge extension extending from a first extended end to a second extended end.

FIG. 14 shows a side view of an exemplary clip spring having a hinge extension extending from the first extended end to the second extended end.

FIG. 15 shows a cross-sectional view of the exterior clip component shown in FIG. 9, with the clip spring extending from a first clip portion to a second clip portion.

FIG. 16 shows a side view of a clip portion component having a hinge protrusion and a hinge receiver on the engagement surface 65.

FIG. 17 shows a side view of an exemplary sleep training device having an exterior clip component with two clip portions and an interior component having a flange configured on an extended end of a flange hub.

FIG. 18 shows a side view of the exemplary sleep training device shown in FIG. 17, with the exterior clip component detachably attached to the interior component.

FIG. 19 shows a perspective view of the exterior clip component shown in FIGS. 17 and 18 having two clip portions, each with a clip handle to actuate the exterior clip component from a closed configuration as shown, to an open configuration, as shown in FIG. 20.

FIG. 20 shows the exterior clip component shown in FIG. 19 now in an open configuration with the two clip portions actuated about the clip hinge.

FIG. 21 shows a perspective bottom view of the exterior clip component in an open configuration and configured over the interior component.

FIG. 22 shows the exterior clip component shown in FIG. 19 now coupled to the interior component.

FIG. 23 shows a top view of the exterior clip component shown in FIG. 20, in an open configuration.

FIG. 24 shows a top view of the exterior clip component shown in FIG. 19, in a closed configuration.

FIG. 25 shows a top view of the exterior clip component decoupled with the first clip component separated from the second clip component.

FIGS. 26 to 30 shows embodiments of a sleep training device comprising an actuator for attaching and detaching the exterior clip component to the interior component.

FIG. 26 is a cross-sectional perspective view of the exterior clip component detached from the interior clip component.

FIG. 27 is a cross-sectional perspective view of the exterior clip component attached to the interior clip component.

FIG. 28 is a cross-sectional side view of the exterior clip component detached from the interior clip component.

FIG. 29 is a cross-sectional side view of the exterior clip component attached to the interior clip component.

FIG. 30 shows bottom perspective view of the exterior clip component with a portion of the interior clip component attached thereto.

FIG. 31 shows an exploded view of an exemplary magnetic sleep training device including a first component having a magnetic component and a second component having a magnetic component configured to magnetically couple with the magnetic component of the first component.

FIG. 32 shows a side view of an exemplary magnetic sleep training device having interface component on both the first component and second component that form a decoupling gap for facilitating detaching the first and second components by hand.

FIG. 33 shows a perspective view of the first component of the magnetic sleep training device shown in FIG. 32.

FIG. 34 an exploded view of the first component of the magnetic sleep training device shown in FIG. 33.

FIG. 35 shows a side view of a sleep training device having a length from a first end to a second end and clips on clip straps on both the first end and second end for detachably attaching to an article of clothing.

FIG. 36 shows an enlarged view of the second end of the sleep training device shown in FIG. 35.

FIG. 37 shows the sleep training device shown in FIG. 35 attached to a garment of a person sleeping.

Corresponding reference characters indicate corresponding parts throughout the several views of the figures. The figures represent an illustration of some of the embodiments of the present invention and are not to be construed as limiting the scope of the invention in any manner. Some of the figures may not show all of the features and components of the invention for ease of illustration, but it is to be understood that where possible, features and components from one figure may be included in the other figures. Further, the figures are not necessarily to scale, some features may be exaggerated to show details of particular components. Therefore, specific structural and functional details disclosed herein are not to be interpreted as limiting, but merely as a representative basis for teaching one skilled in the art to employ the present invention.

### DETAILED DESCRIPTION OF THE ILLUSTRATED EMBODIMENTS

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

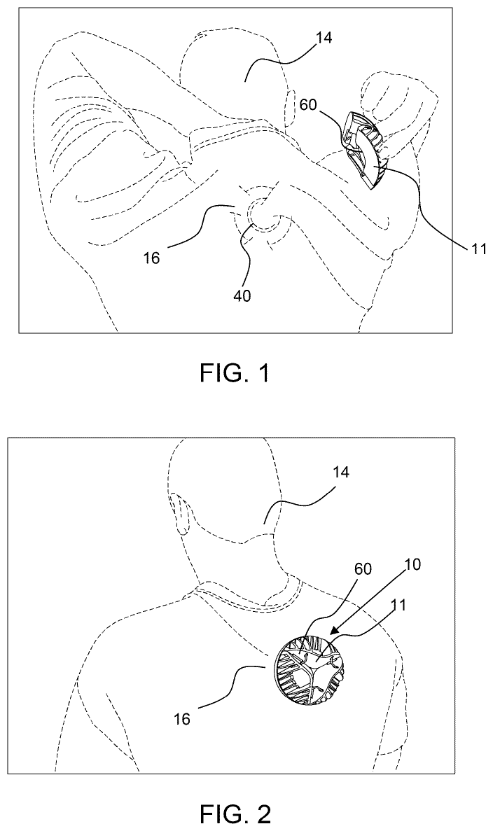

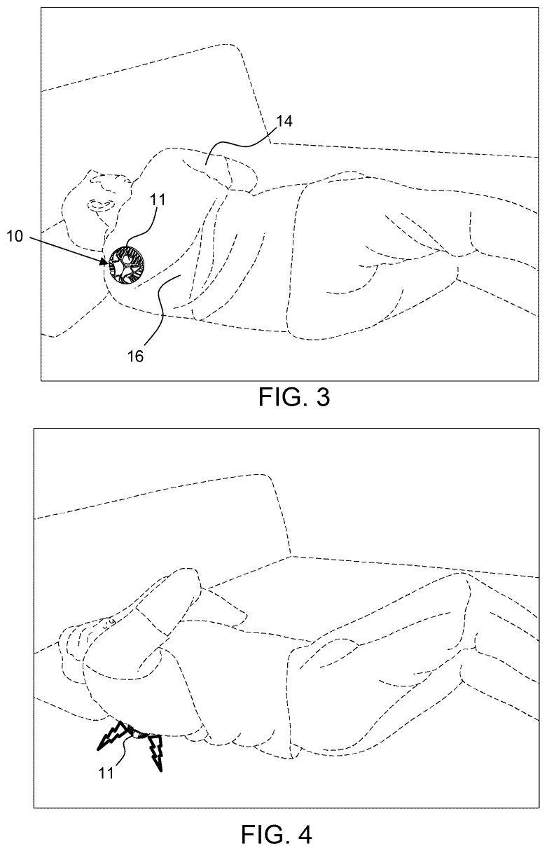

Referring now to FIGS. 1 to 4, an exemplary sleep training system 10 employs a sleep training device 11 configured for detachable attachment to a garment 16 by clipping an exterior clip component 60 to an interior component 40. The interior component is configured inside the garment 16 and the exterior clip component is actuated and then clipped onto the interior component. In FIG. 1, the person has actuated the exterior clip component to an open configuration by hand, and is retaining the interior component 40 inside of the garment 16. As shown in FIG. 2, the sleep training device 11 is attached to the back portion of a garment 16 and is protruding from the person's back. As shown in FIG. 3, the person 14 is sleeping on their side with the sleep training device 11 of the sleep training system 10 coupled to the back portion of a garment 16. As shown in FIG. 4, the person is rolling onto their back, wherein the sleep training device 11 presses on the person's back to stop them from rolling onto their back.

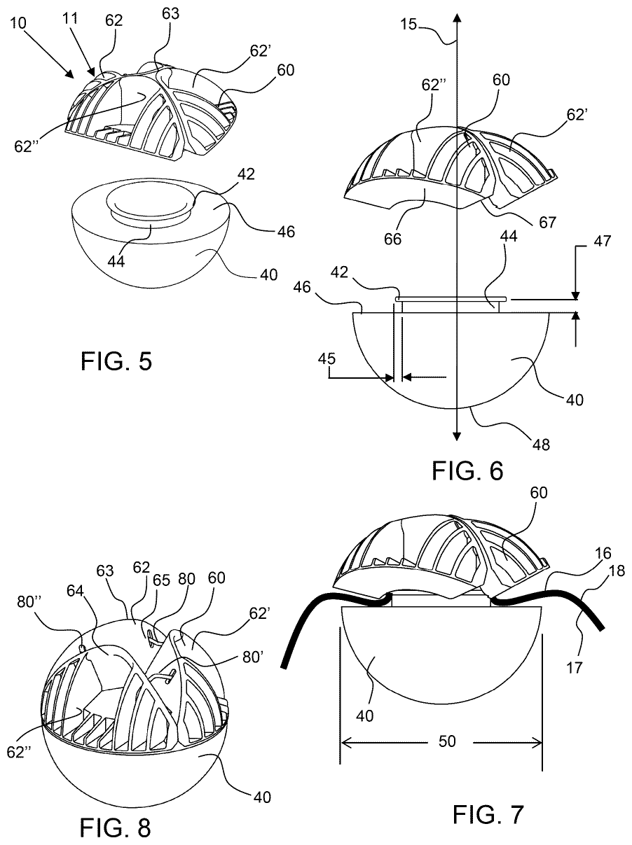

Referring now to FIGS. 5 to 15, an exemplary sleep training system 10 includes a sleep training device 11 having an interior component 40 with a flange 42 extending from a flange hub 44, and an exterior clip component 60 configured to actuate to open a flange aperture 67 on a flange interface surface 66 to couple the exterior clip component to the flange of the interior component. As shown in FIG. 7, the exterior clip component 60 is on an outside surface 18 of the garment 16 and the interior component 40 is on an inside surface 17 of the garment 16.

The interior component 40 has a flange 42 extending a flange extension distance 45 and configured a flange offset distance 47 from the clip interface surface 46 of the interior component 40. The flange 42, as shown in FIG. 6, is configured on the extend end of the flange hub 44. The interior component has a distal surface 48 opposite the interface surface 46 that is configured to contact a person's back when the sleep training device 11 is detachably attached to a garment and said garment is donned for sleeping. The distal surface is a pressure surface or surface that applies pressure to the person's back while sleeping. As shown in FIG. 6, the distal surface 48 extends along a radius of curvature and the interior component may be spherical in shape along the pressure surface and may form a partial or half sphere as shown. The flange hub extends up along the attachment axis 15, which is orthogonal to the interface surface 46.

The exterior clip component 60 has three clip portions 62, 62′, 62″ that are coupled together by clip springs 80, 80′, 80″, shown in FIG. 8. Each of the clip portions 62, 62′, 62″ have a respective clip handle 64, 64′, 64″ as shown in FIG. 11. These handles may be squeezed or actuated inward toward a centerline 61 or center point of the exterior clip component to open the flange aperture 67. As shown in FIG. 10, the handles are forced radially outward from the centerline 61 by the clip springs and in FIG. 11, the handles are actuated inward toward the centerline.

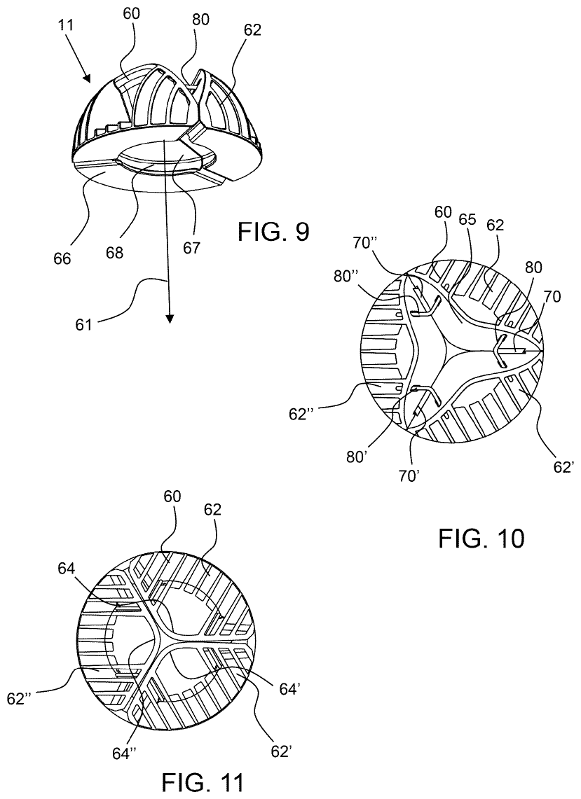

The exterior clip component 60 has a flange receiver 68, as shown in FIG. 9 that may be a recess or groove within the flange aperture 67. The clip portions are coupled together by clip springs 80, 80′, 80″ that extend between adjacent clip portions to force the clip portions and exterior clip component to a closed position, to retain the exterior clip component to the interior component.

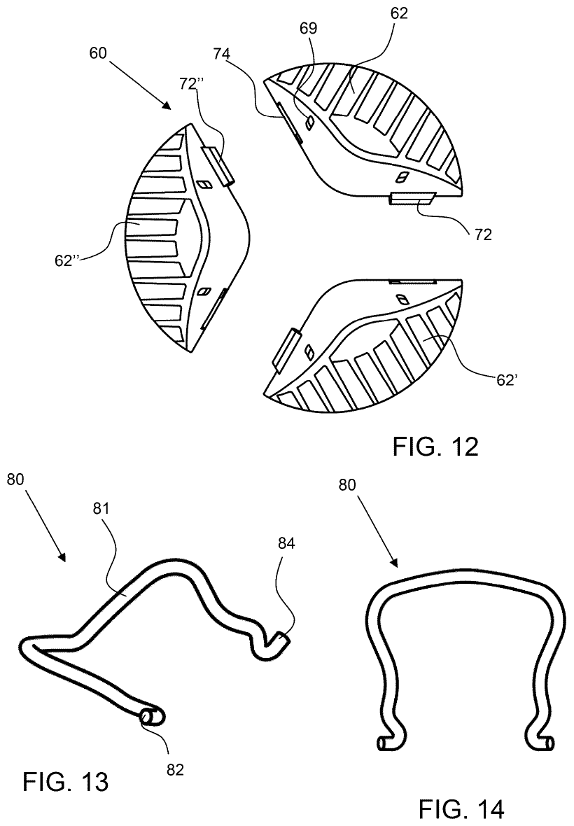

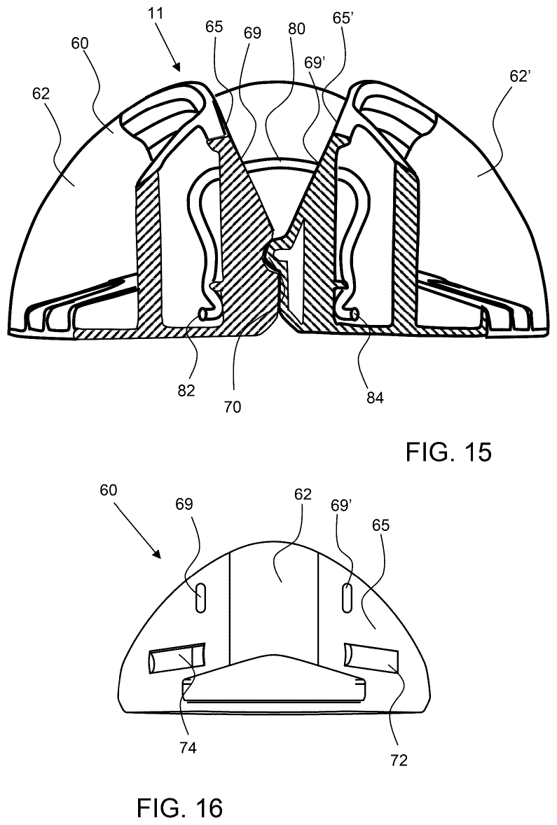

As shown in FIG. 12, the clip portions have a hinge protrusion 72 on a first side and a hinge receiver 74′ on a second side of the engagement surface 65 of the clip component 62. The hinge protrusion 72″ of clip portion 62″ is configured for coupling with the hinge receiver 74 of clip portion 62 to form a hinge. Also, each clip portion has a spring receiver 69, such as a recess, flange or aperture to retain the clip spring 80, as shown in FIG. 15. The spring receivers 69, 69′ of clip portion 62, 62′ may be an aperture for insertion of a first extended end 82 of the clip spring 80 and spring receiver 69″ may be an aperture for receiving the second extended end 84 of the clip spring 80.

Referring now to FIGS. 10, 11, and 12, the exterior clip component 60 has three clip portions 62, 62′, 62″ that are hingeably engaged by hinges 70, 70′, 70″ and have clip springs 80, 80′, 80″ coupling together the three clip portions. In FIG. 10, the exterior clip component is in a closed configuration with the springs extended and in FIG. 11 the exterior clip component is in an open configuration with the springs compressed or flexed to bring each of the engagement surfaces proximal to the extended end of the clip portions toward each other. The engagement surface 65 is an interior surface that extends to an extended end 63 (shown in FIG. 5) most distal form the flange interface surface 66. The exterior clip component may also have a spherical shape wherein each of the clip portions from a wedge shape of a half spherical shape. When the interior component and the exterior clip component are attached, the sleep training device may be a full spherical shape having a diameter 50 (shown in FIG. 7) and as described herein.

Referring now to FIGS. 13 to 16, the clip portions are coupled together about the hinge by clip springs 80. A clip spring extends between two adjacent clip portions and has a spring extension 81 that extends from a first extended end 82 to a second extended end 84. As shown in FIG. 15, the clip spring 80 extends between two adjacent clip portions. The spring extension 81 extends through the respective spring receiver apertures 69, 69′ in the engagement surfaces 65, 65′ of the first and second clip portions 62, 62′. As shown in FIG. 16, the clip portion 62 has a hinge protrusion 72 and a hinge receiver 74, such as a recess, on the engagement surface 65 for coupling with the other two clip portions, not shown.

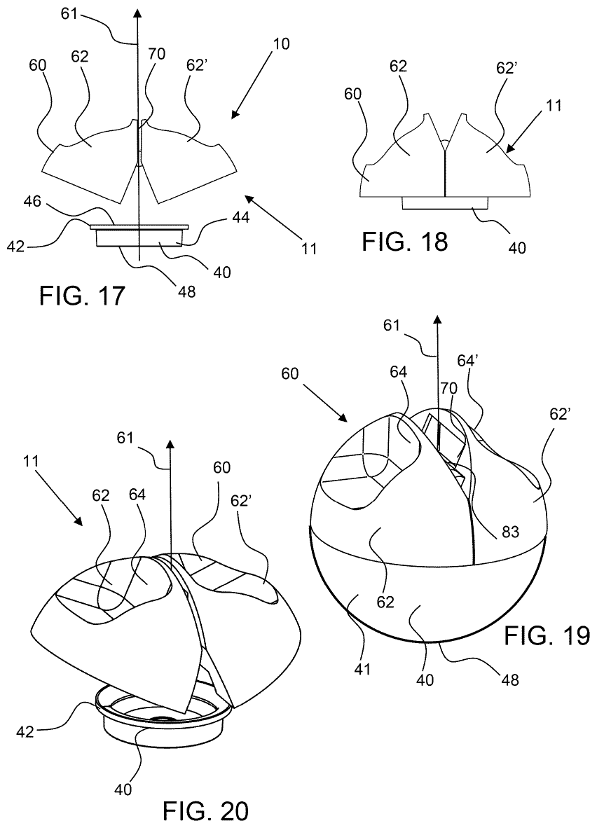

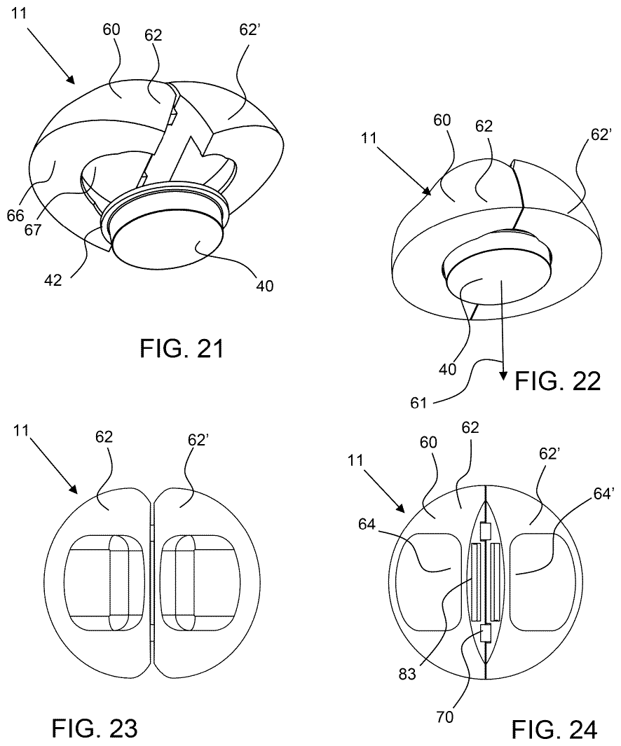

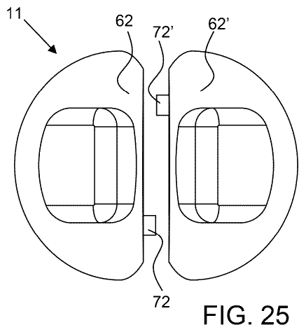

Referring now to FIGS. 17 to 25, an exemplary sleep training system 10 includes a sleep training device 11 having an interior component 40 with a flange 42 and an exterior clip component 60 with two clip portions 62, 62′ that are configured to actuate to open a flange aperture 67 on a flange interface surface 66 to couple the exterior clip component to the flange of the interior component.

The interior component has a flange 42 extending a flange extension distance and configured a flange offset distance from the clip interface surface 46 of the interior component 40. The flange 42 as shown in FIG. 17 is configured on the extend end of the flange hub 44. The interior component has a distal surface 48 opposite the interface surface and configured to contact a person's back when the sleep training device 11 is detachably attached to a garment and said garment is donned for sleeping. The distal surface is a pressure surface or surface that applies pressure to the person's back while sleeping. The distal surface 48 of the interior component 40 and/or the exterior clip component 60 may extend along a radius of curvature. The interior component 40 shown in FIGS. 17 to 25 may have a body portion having a curved distal surface that is detachably attachable to the interior component.

The exterior clip component 60 has two clip portions 62, 62′ that are coupled together by a clip spring. Each of the clip portions 62, 62′, have a respective clip handle 64, 64′, as shown in FIG. 19 and FIG. 24. These handles may be squeezed or actuated inward toward a centerline 61 or center point of the exterior clip component to open the flange aperture 67. As shown in FIG. 19, the handles are forced radially outward from the centerline 61 by the clip spring 83 and in FIG. 20, the handles are actuated inward toward the centerline. The clip spring 83 may be magnets that oppose each other to keep the two clip portions 62, 62′ of the exterior clip component closed, or may be a coiled spring or an elastomeric material to force these two components apart. An interior component body 41 is not shown in FIGS. 17, 18, 20 and 21-25 but may include a spherical extension as shown in FIG. 19. As shown in FIG. 19, the distal surface 48 of the interior component 40 extends along a radius of curvature and the interior component may be spherical in shape along the pressure surface and may form a partial or half sphere as shown.

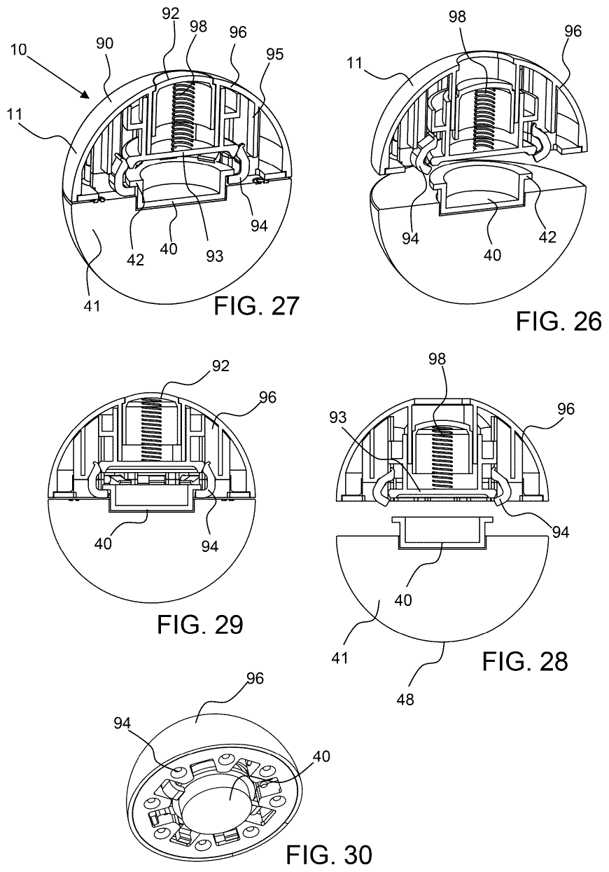

Referring now to FIGS. 26 to 30, an exemplary sleep training system 10 includes a sleep training device 90 having an interior component 40 with a flange 42 and an exterior clip component 96 with an actuator 92 such as an actuator button as shown for attaching and detaching the exterior clip component to the interior component. The actuator button is coupled with latches 94 that are actuated to an open position when the actuator button is pressed. The actuator button moves an actuator interface 93 that interfaces with the lathes 94 to move from a closed to an open position. A return spring 98 moves the actuator button back to a ready position. The actuator button moves into the exterior clip body 95 when actuated to move the latch to an open position. As shown in FIG. 28, the distal surface 48 of the interior component 40, or interior component body 41 extends along a radius of curvature and the interior component may be spherical in shape along the pressure surface and may form a partial or half sphere as shown.

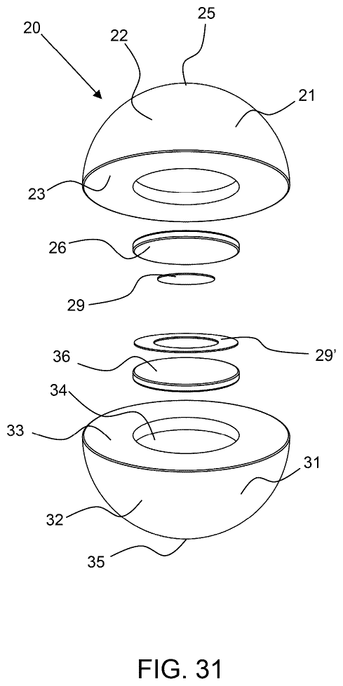

FIG. 31 shows an exploded view of an exemplary magnetic sleep training device 20 includes a first component 22 having a first magnetic component 26 and a second component 32 having a second magnetic component 36 configured to magnetically couple with the magnetic component of the first component. The first component body 21 and the second component body 31 may have a recess 34 (shown in the second component body) to receive the respective magnetic component. The magnets are configured on the interface surface 23, 33 of the first and second components 22, 32 respectively. The distal surface 25, 35 of the first and second components are rounded and each of the first and second components have a spherical shape, a half of a sphere that forms a full sphere when attached by the magnets on the interface surfaces. Stabilizing components 29, 29′ may be configured between the first magnetic component 26 and the second magnetic component 36 to stabilize the connection and prevent sliding and to also make it easier to decouple the first and second magnets from each other to remove the magnetic sleep training device 20 from a garment. The stabilizing component 29 is configured to seat within the stabilizing component 29′, a ring of material. Also, the first component body 21 and second component body 31 may be made of a resilient material, such as a foam, such as a polyurethane foam and may be a high density polyurethane foam.

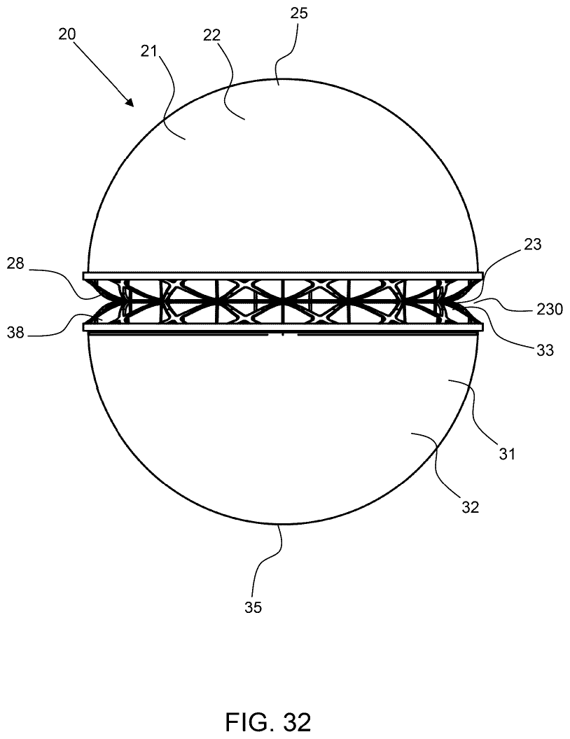

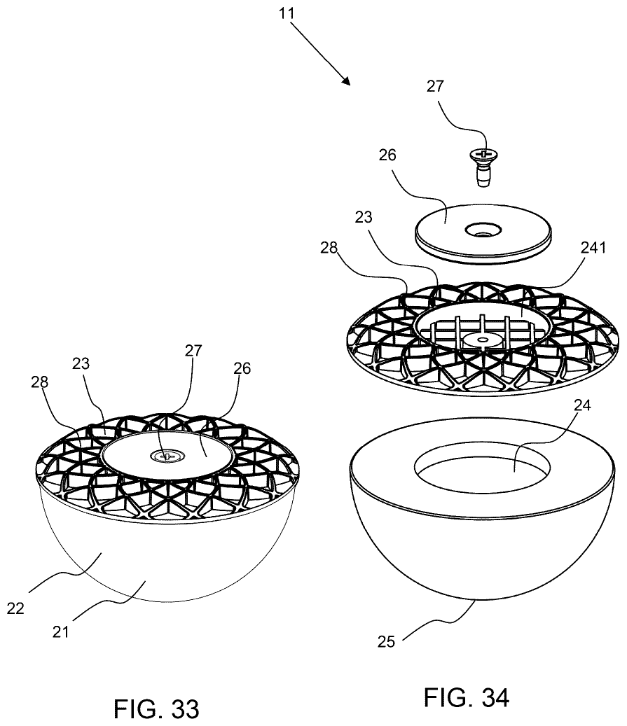

Referring now to FIGS. 32 to 34, an exemplary magnetic sleep training device 20 includes a first component 22 having a magnetic component 26 and a second component 32 having a magnetic component 36 configured to magnetically couple with the magnetic component of the first component. The first component 22 has an interface component 28 and the second component 32 has an interface component 38 configured to retain the respective first magnet 26 and second magnet 36. As shown in FIG. 34, the magnetic component 26 is retained in the first interface component 28 by a fastener 27 and the first interface component has a recess 241 for receiving the magnetic component therein. The first component body 21 also has a recess 24 for receiving the protrusion from the first interface component 28. The first interface component 28 and second interface component 38 form the respective interface surfaces 23, 33 of the first and second component 22, 32 and make it easier to detach the first component from the second component 32. As shown in FIG. 32, the first and second interface components 28, 38 form a decoupling gap 230 that enables a person to insert their finger to pry apart the two components to detach the magnetic sleep training device 20 from a garment. A magnetic component may be a magnet, such as a permanent magnet, or a material that is magnetically attracted to a magnet, such a steel or other metal that is attracted to a permanent magnet.

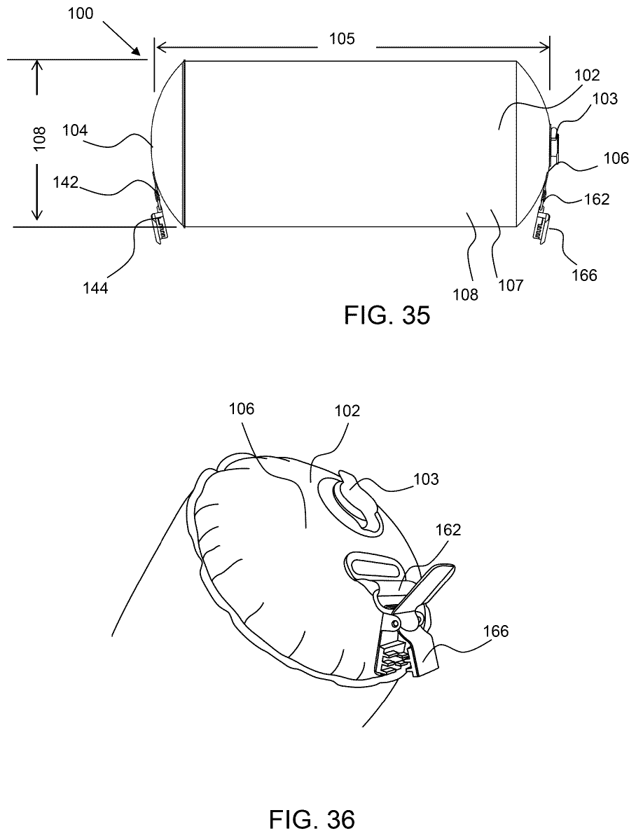

Referring now to FIGS. 35 to 37, a sleep training device 100 has a length 105 from a first end 104 to a second end 106 and respective clips 144, 166 on clip straps 142, 162 on both the first end and second end for detachably attaching to an article of clothing. The clip straps enable the clips to couple with a garment while the clip strap is positioned more centrally on the end of the sleep training device. The sleep training body 102 may be an elongated body that is cylindrical in shape having a diameter 108 and length 105. The sleep training device may be inflatable having an inflation port 103 that fills a bladder 107. A cover 109 may be configured around the bladder for comfort and may include a fabric or a flocking material coupled to the bladder.

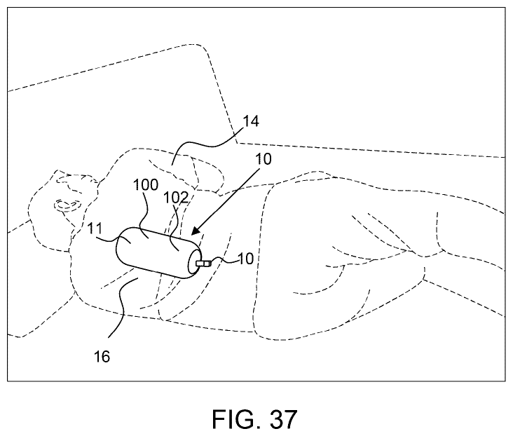

As shown in FIG. 37 the elongated body may be configured with the length extending along the person's spine or back with a first end proximal to their head and the opposing second end more proximal to the buttock.

It will be apparent to those skilled in the art that various modifications, combinations and variations can be made in the present invention without departing from the scope of the invention. Specific embodiments, features and elements described herein may be modified, and/or combined in any suitable manner. Thus, it is intended that the present invention covers the modifications, combinations and variations of this invention provided they come within the scope of the appended claims and their equivalents.

What is claimed is:

1. A sleep training system comprising:
a) a garment having an interior side and an exterior side;
b) a sleep training device comprising:
i) an interior component comprising:
ii) an exterior clip component comprising:
wherein the exterior clip component is configured to detachable attach to the interior component to secure the sleep training device to said garment with the interior component configured on said interior side of said garment and with the exterior clip component configured on said exterior side of said garment.

2. The sleep training system of claim 1, wherein the exterior clip component comprises:
a) a first clip portion and a second clip portion; and
b) a first clip hinge, wherein the first clip portion and second clip portion are hingeably coupled about said clip hinge.

3. The sleep training system of claim 2, wherein the exterior clip component further comprises a clip spring extending between the first clip portion and the second clip portion and configured to force the exterior clip component to a closed position.

4. The sleep training system of claim 3, wherein the interior component comprises a flange.

5. The sleep training system of claim 4, wherein the flange is configured on an extended end of a flange hub.

6. The sleep training system of claim 4, wherein the exterior clip component further comprises a flange aperture configured on a flange interface surface, and wherein the flange aperture is configured to extend over said flange of the interior component to detachably attach the exterior clip portion.

7. The sleep training system of claim 6, wherein the exterior clip component further comprises a flange receiver configured to extend around the flange of the interior component to secure the sleep training device to said garment with the interior component configured on an interior side of said garment and with the exterior clip component configured on an exterior side of said garment.

8. The sleep training system of claim 7, wherein the first clip portion and a second clip portion of the exterior clip component are configured to actuate about the first clip hinge to an open configuration to expand the flange aperture for clipping the exterior clip component to the interior component with the flange receiver extending around the flange and wherein the clip spring is configured to retain the exterior clip component to the interior component.

9. The sleep training system of claim 8, wherein the exterior clip component has a distal surface that is curved.

10. The sleep training system of claim 9, wherein the distal surface of the exterior clip component extends along a radius of curvature.

11. The sleep training system of claim 9, wherein the interior component has a distal surface that is curved.

12. The sleep training system of claim 11, wherein the distal surface of the interior component extends along a radius of curvature.

13. The sleep training system of claim 11, wherein the distal surface of the exterior clip component extends along a radius of curvature.

14. The sleep training system of claim 13, wherein the sleep training device is spherical in shape with the exterior clip component attached to the interior component.

15. The sleep training system of claim 14, wherein the sleep training device has a diameter of at least 50 mm to about 200 mm.

16. The sleep training system of claim 1, wherein the interior component comprises a flange.

17. The sleep training system of claim 16, wherein the flange is configured on an extended end of a flange hub.

18. The sleep training system of claim 17, wherein the interior component has a clip interface surface and wherein the flange hub extends from said clip interface surface.

19. The sleep training system of claim 1, wherein the exterior clip component has an actuator that is actuated to attach the exterior clip component to the interior component to secure the sleep training device to said garment with the interior component configured on said interior side of said garment and with the exterior clip component configured on said exterior side of said garment.

20. The sleep training system of claim 19, wherein the actuator comprises an actuator button that is coupled with latches that are moved from a closed configuration to an open configuration when the actuator button is pressed.
